(19) United States
(12) Patent Application Publication
(10) Pub. No.: US 2022/0064516 A1
(43) Pub. Date: Mar. 3, 2022

(54) Materials and Methods for Extended Reduction of Heavy Crude Oil Viscosity

(71) Applicant: Locus Oil IP Company, LLC, Solon, OH (US)

(72) Inventors: Sean FARMER, Ft. Lauderdale, FL (US); Ken ALIBEK, Solon, OH (US); Anthony NERRIS, Solon, OH (US); Blake OTT, Solon, OH (US)

(21) Appl. No.: 17/420,920

(22) PCT Filed: Jan. 2, 2020
(86) PCT No.: PCT/US2020/012042

§ 371 Date: Jul. 6, 2021

Related U.S. Application Data
(60) Provisional application No. 62/787,887, filed on Jan. 3, 2019.

(51) Int. Cl. C09K 8/582; C12N 1/16; C12N 1/14; C12N 1/20; C10G 71/00; E21B 43/16

(52) U.S. Cl. CPC C09K 8/582; C12N 1/16; C12N 1/14; C10G 2300/302; C10G 71/00; E21B 43/16; C12N 1/20

(57) ABSTRACT

The present invention provides environmentally-friendly compositions and methods for reducing the viscosity of crude oil using a mixture of microorganisms and/or metabolites produced by microorganisms The compositions and methods provide for efficient reduction in the viscosity of heavy crude oils, which, advantageously, endures for extended periods of time.

### CROSS-REFERENCE TO RELATED APPLICATION

This application claims priority to U.S. Provisional Patent Application No. 62/787,887, filed Jan. 3, 2019, which is incorporated herein by reference in its entirety.

### BACKGROUND OF THE INVENTION

The high demand for fossil fuels necessitates efficient production of oil. A number of challenges in the production of oil derive from the viscosity, surface tension, hydrophobicity and density of crude oil.

Some crude oils have naturally higher viscosities than others. Heavy and extra heavy crude oils are highly viscous with a density close to or even exceeding water. Heavy oils are crudes that have API gravity less than 20° or viscosity higher than 200 centipoises (cp). Extra heavy oil refers to petroleum with API gravity less than 12° and viscosity higher than 10,000 cp (“Heavy Oil” 2016). Extra-heavy crude oil can be heavier than water and, therefore, can sink to the bottom of a water formation, causing subsurface contamination.

On the other hand, “light” crude oil, or that which has low density and which flows freely at room temperature, has low viscosity and high API gravity due to its higher proportion of light hydrocarbon fractions. Low viscosity crude oils can weather over time into more viscous liquids.

Heavy and extra heavy crude oils are a major potential energy resource. Forty percent of the world's total oil reserves are heavy and extra heavy oil, accounting for 3.6-5.2 trillion bbl of oil. Thus, recovery of these highly viscous hydrocarbons could have major economic significance. However, most heavy and extra heavy oils, asphalts, tars and/or bitumens are highly viscous, and thus, burdensome to transport using conventional methods, such as portable storage tanks and tanker trucks. A significant amount of energy is required to pump oil with higher viscosity through pipelines to refineries and processing facilities.

Heavy oil is also difficult to extract from the ground, due to its viscosity, hydrophobicity and immiscibility with water. Viscosity, in particular, affects the speed at which crude oil can be pumped from a reservoir, with more viscous oils contributing to a decrease in overall productivity for an oil field.

The properties of crude oil also contribute to the difficulty of environmental remediation following, for example, an oil spill onto a body of water. The high interfacial tension causes oil to float on water and adhere to plants, animals and soil. As the aromatic constituents of the oil evaporate, the heavier residues can sink, thereby causing subsurface contamination. Current treatment of spilled oil on water surfaces relies on time-consuming and expensive methods for degrading the oil.

One method of maintaining the flowability of heavy hydrocarbons is to keep them at elevated temperatures. Another well-known method is to mix the heavy oil with a lighter hydrocarbon diluent. This helps to enable, for example, pipeline transportation of the oil. Nonetheless, diluents can be expensive to obtain and transport to oil fields.

Surfactants have also been widely used in the petroleum industry to ameliorate a number of the negative physical properties of crude oil. Surfactant molecules consist of hydrophobic and hydrophilic parts. Their amphiphilic nature allows them to be adsorbed at an oil/water interface, forming micelles that reduce the interfacial tension between the oil and water. The use of chemicals in oil production, however, can result in costs to safety and the environment, as well as for producing and/or obtaining these chemicals.

The use of microorganisms and/or their growth by-products, such as, for example, biosurfactants, has also been used in recent years. However, the effectiveness of these methods, particularly over extended periods of time, has been unpredictable and unreliable.

Efficient production of oil and gas is crucial to meet the high demand for such products. Because of the importance of safe and efficient oil and gas production, the difficulties of producing and transporting heavy crude oil, and the untapped potential of heavy oils to be converted into useful products, there is a continuing need for methods of improving the physical properties of heavy oil, particularly by reducing its viscosity.

### BRIEF SUMMARY OF THE INVENTION

The subject invention provides environmentally-friendly, cost-efficient materials and methods for enhancing the recovery and improving the transportation of oil. In specific embodiments, the subject invention provides microbe-based compositions and methods for reducing viscosity of heavy crude oil.

In certain embodiments, the subject invention provides materials and methods for improving oil production by treating oil-containing sites with a microbe-based composition capable of reducing the viscosity of oil. Advantageously, the subject compositions and methods can be used to improve the viscosity, and/or enhance recovery, of heavy crude oil in “mature” or even “dead” oil reservoirs.

In preferred embodiments, the microbe-based composition of the present invention comprises one or more cultivated microorganisms and/or microbial growth by-products, such as biosurfactants, solvents, and/or enzymes. The subject invention also provides methods of using these microbes and their by-products.

The one or more microorganisms can comprise yeasts, fungi and/or bacteria. In one embodiment, the composition comprises a yeast, a fungus and a bacterium.

In one embodiment, the composition comprises a Pichia yeast, such as, for example, P. occidentalis or P. kudriavzevii. In a specific embodiment, the yeast is a unique strain of P. occidentalis that was selected for enhanced enzymatic activity and viscosity-reducing capabilities.

In one embodiment, the composition comprises a Trichoderma fungus, such as, for example, T. harzianum. In one embodiment, the composition comprises a Cronobacter bacterium, such as, for example, C. sakazakii.

In one embodiment, the one or more microorganisms comprise, consist of, or consist essentially of a mixture of Pichia occidentalis, Trichoderma harzianum, and Cronobacter sakazakii.

In one embodiment, the composition is obtained through cultivation processes ranging from small to large scale. The cultivation process can be, for example, submerged cultivation, solid state fermentation (SSF), and/or any hybrid, modification, or combination thereof.

The composition of the subject invention can also comprise additional components, including, for example, surfactants, emulsifiers, enzymes, solvents, acids, and other additives. These components can be chemical or cell-derived (e.g., from microbial or plant cells).

In a specific embodiment, an organic solvent, such as primary amyl acetate, is included in the composition.

In one embodiment the subject invention provides a method for improving oil recovery by applying to heavy oil, or to an oil recovery site containing heavy oil, the microbe-based composition comprising one or more strains of microorganisms and/or microbial growth by-products.

In one embodiment, the method optionally includes adding nutrients and/or other agents to the site in order to, for example, promote microbial growth.

The microbes can be live (or viable), in spore form, or inactive at the time of application. In preferred embodiments, different microbe strains are cultivated separately, then mixed together prior to, or at the time of, application to the heavy crude oil or oil recovery site.

The crude oil can be incubated with the composition for, e.g., 1 day or longer. The viscosity of crude oil can be decreased by, for example, 20 to 60%, and remain at a decreased level for extended periods of time, for example, as long as two weeks (14 days) or longer. Compared with other methods, which often result in a return of the crude oil to its heavy, viscous state shortly after treatment, e.g., overnight, the subject invention provides enhanced methods for improving the characteristics of heavy oil, as well as improving its recovery and/or transportation.

In one embodiment, the method further comprises the step of subjecting the heavy oil to cavitation either immediately prior to, simultaneously with, and/or sometime after the microbe-based composition has been applied to the heavy oil or oil recovery site. The cavitation can be carried out using machinery known in the art, and can comprise, for example, hydrodynamic or ultrasonic methods.

The microorganisms of the subject invention can reduce the viscosity of heavy crude oil by, for example, 20% or more due to, for example, direct consumption and/or degradation of the heavy hydrocarbon molecules, and/or the production of metabolites that act upon the heavy oil to reduce its viscosity.

The microorganisms can grow in situ and produce active compounds onsite. Consequently, a high concentration of metabolites and/or the microorganisms that produce them at a treatment site (e.g., an oil well) can be achieved easily and continuously.

In one embodiment, the present invention allows for more efficient transportation of oil. For example, once the viscosity of heavy oil is reduced, it can be transported by pipeline rather than requiring storage tanks and/or transportation via trucks.

The methods and microbe-based products of the subject invention can be used in a variety of unique settings because of, for example, the ability to efficiently deliver: 1) fresh fermentation broth with active metabolites; 2) a mixture of cells, spores and/or mycelia and fermentation broth; 3) a composition with vegetative cells, spores and/or mycelia; 4) compositions with a high density of cells, including vegetative cells, spores and/or mycelia; 5) microbe-based products on short-order; and 6) microbe-based products in remote locations.

Advantageously, the present invention can be used without releasing large quantities of inorganic compounds into the environment. Additionally, the compositions and methods utilize components that are biodegradable and toxicologically safe. Thus, the present invention can be used in all possible operations of oil and gas production as a “green” treatment.

### DETAILED DESCRIPTION

The subject invention provides advantageous uses for microbes, as well as the by-products of their growth. In certain embodiments, the subject invention provides microbe-based products, as well as their uses in improved oil production. In specific embodiments, the methods and compositions described herein utilize microorganisms to improve the quality of oil by reducing its viscosity.

### Selected Definitions

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In preferred embodiments, the microbes are present, with broth in which they were grown, in the microbe-based composition. The cells may be present at, for example, a concentration of 1×10⁴, 1×10⁵, 1×10⁶, 1×10⁷, 1×10⁸, 1×10⁹, 1×10¹⁰, or 1×10¹¹ or more propagules per milliliter of the composition. As used herein, a propagule is any portion of a microorganism from which a new and/or mature organism can develop, including but not limited to, cells, spores, conidia, mycelia, buds and seeds.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “harvested” refers to removing some or all of the microbe-based composition from a growth vessel.

As used herein, a “biofilm” is a complex aggregate of microorganisms, such as bacteria, wherein the cells adhere to each other on a surface. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. As used herein, reference to “isolated” in the context of a microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state.

A “metabolite” refers to any substance produced by metabolism or a substance necessary for taking part in a particular metabolic process. A metabolite can be an organic compound that is a starting material (e.g., glucose), an intermediate (e.g., acetyl-CoA) in, or an end product (e.g., n-butanol) of metabolism. Examples of metabolites can include, but are not limited to, enzymes, toxins, acids, solvents, alcohols, proteins, carbohydrates, vitamins, minerals, microelements, amino acids, polymers, and surfactants.

By “modulate” is meant alter (e.g., increase or decrease). Such alterations are detected by standard art known methods such as those described herein.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 1%, 5%, 10%, 25%, 50%, 75%, or 100%.

By “reference” is meant a standard or control condition.

By “salt-tolerant” is meant a microbial strain capable of growing in a sodium chloride concentration of fifteen (15) percent or greater. In a specific embodiment, “salt-tolerant” refers to the ability to grow in 150 g/L or more of NaCl.

By “surfactant” is meant a compound that lowers the surface tension (or interfacial tension) between two liquids or between a liquid and a solid. Surfactants act as detergents, wetting agents, emulsifiers, foaming agents, and dispersants.

As used herein, “applying” a composition or product refers to contacting it with a target or site such that the composition or product can have an effect on that target or site. The effect can be due to, for example, microbial growth and/or the action of a biosurfactant or other growth by-product. For example, the microbe-based compositions or products can be injected into oil wells and/or the piping, pumps, tanks, etc. associated with oil wells and oil processing.

As used herein, “heavy oil” or “heavy hydrocarbons” mean viscous hydrocarbon fluids. Heavy hydrocarbons may include highly viscous hydrocarbon fluids such as heavy oil, extra heavy oil, tar, tar sands, fuel oil and/or asphalt. Heavy and extra heavy oils are highly viscous with a density close to or even exceeding water. Heavy hydrocarbons may comprise moderate to high quantities of paraffins, resins and asphaltenes, as well as smaller concentrations of sulfur, oxygen, and nitrogen. Heavy hydrocarbons may also include aromatics or other complex ring hydrocarbons. Additional elements may also be present in heavy hydrocarbons in trace amounts. Heavy hydrocarbons may be classified by API gravity. Heavy hydrocarbons generally have an API gravity below about 20°. Heavy oil, for example, generally has an API gravity of about 10-20°, whereas extra heavy oil generally has an API gravity below about 12°. The viscosity of heavy hydrocarbons is generally greater than about 200 cp at reservoir conditions, and that of extra heavy oil is generally about 10,000 cp or more.

As used herein, “upgrading” or “converting” or “improving the quality of” or “increasing the value of” heavy oil and/or hydrocarbons means changing the structure of the hydrocarbons and/or the contents of the oil in such a way as to increase its overall utility to consumers, and thus, its value to producers. For example, the Btu, i.e., energy or heat content, of the oil can be increased, thus increasing the value of heavy crude before it is sold to refineries. This can also benefit oil refineries who can buy cheaper heavy crude and convert it to a more usable product, such as, for example, road asphalt, using the subject methods and compositions. Upgrading can also involve increasing the API gravity, reducing viscosity, and/or reducing the impurities content of heavy hydrocarbons. Impurity is often a free radical that attaches to large hydrocarbon molecules. Typical impurities found in heavy oil can include, for example, sulfur or hydrogen sulfide, ash, nitrogen, heavy metals, olefins, aromatics, naphthenes, and asphaltenes.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example, within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Microbe-Based Compositions

The subject invention provides environmentally-friendly, cost-efficient materials and methods for enhancing the recovery and improving the transportation of oil. In specific embodiments, the subject invention provides microbe-based compositions and methods for reducing viscosity of heavy crude oil.

The composition can be used to convert heavy oil to light oil. The composition can further be used to enhance oil recovery, including recovery of oil from oil sands. Furthermore, the composition can be used to improve the transportation of oil by allowing for transport via pipelines rather than storage and transportation tanks.

In preferred embodiments, the microbe-based composition of the present invention comprises one or more cultivated microorganisms and/or microbial growth by-products, such as biosurfactants, solvents, and/or enzymes.

The one or more microorganisms can comprise yeasts, fungi and/or bacteria. In one embodiment, the composition comprises a yeast, a fungus and a bacterium. The ratio of each microbe in the composition can be either 1:1:1 or some other combination based upon which microbes are included.

In some embodiments, the microbes used according to the subject invention are “over-producers” of a particular desirable metabolite, such as, for example, an enzyme, solvent or biosurfactant. For example, the microbes can produce at least 10%, 25%, 50%, 100%, 2-fold, 5-fold, 7.5 fold, 10-fold, 12-fold, 15-fold or more compared to other microbial strains.

In one embodiment, the composition comprises a Pichia yeast, such as, for example, P. occidentalis or P. kudriavzevii. In a specific embodiment, the yeast is a unique strain of P. occidentalis that was selected for enhanced enzymatic activity (i.e., over-production of enzymes) and viscosity-reducing capabilities.

In one embodiment, the composition comprises a Trichoderma fungus, such as, for example, T. harzianum. Trichoderma can produce useful metabolites, such as, for example, glycolipid biosurfactants, to help with reduction of oil viscosity.

In one embodiment, the composition comprises a Cronobacter bacterium, such as, for example, C. sakazakii. Cronobacter spp. have been indicated as having certain hydrocarbon-degradation capabilities.

In one embodiment, the one or more microorganisms comprise, consist of, or consist essentially of a mixture of Pichia occidentalis, Trichoderma harzianum, and Cronobacter sakazakii.

The microbe-based composition can comprise the fermentation medium containing a live culture and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

Advantageously, in accordance with the subject invention, the microbe-based composition may comprise growth medium in which the microbes were grown. The product may be, for example, at least, by weight, 1%, 5%, 10%, 25%, 50%, 75%, or 100% growth medium. The amount of biomass in the product, by weight, may be, for example, anywhere from 0% to 100% inclusive of all percentages therebetween.

In the case of submerged fermentation, the biomass content of the fermentation broth may be, for example from 5 g/l to 180 g/l or more. In one embodiment, the solids content of the broth is from 10 g/l to 150 g/l.

Further components can be added to the microbe-based composition, for example, buffering agents, carriers, other microbe-based compositions produced at the same or different facility, viscosity modifiers, preservatives, nutrients for microbe growth, tracking agents, biocides, other microbes, surfactants, emulsifying agents, lubricants, solubility controlling agents, pH adjusting agents, preservatives, stabilizers and ultra-violet light resistant agents.

In certain embodiments, the composition comprises, for example, surfactants, emulsifiers, enzymes, solvents, acids, and other additives. These components can be chemical or cell-derived (e.g., from microbial or plant cells).

In a specific embodiment, an organic solvent, such as isoamyl acetate or primary amyl acetate, is included in the composition. The concentration of organic solvent can range from, for example, about 10 ml/L to 200 ml/L, about 20 ml/L to 175 ml/L, about 30 ml/L to 150 ml/l, about 40 ml/L to 125 ml/L, or about 50 ml/L to 100 ml/L.

In one embodiment, the composition can further comprise buffering agents, including organic and amino acids or their salts to stabilize pH near a preferred value. Suitable buffers include, but are not limited to, citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and mixtures thereof. Phosphoric and phosphorous acids or their salts may also be used. Synthetic buffers are suitable to be used but it is preferable to use natural buffers such as organic and amino acids or their salts.

In a further embodiment, pH adjusting agents include potassium hydroxide, ammonium hydroxide, potassium carbonate or bicarbonate, hydrochloric acid, nitric acid, sulfuric acid and mixtures thereof The pH of the microbe-based composition should be suitable for the microorganism of interest. In one embodiment, the pH of the microbe-based composition ranges from 7.0-7.5.

In one embodiment, additional components such as an aqueous preparation of a salt as polyprotic acid, such as sodium bicarbonate or carbonate, sodium sulfate, sodium phosphate, or sodium biphosphate, can be included in the microbe-based composition.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

In certain embodiments, use of the microbe-based compositions according to the subject invention can be superior to, for example, purified microbial metabolites alone, due to, for example, the advantageous properties of yeast cell walls. These properties include high concentrations of mannoprotein as a part of yeast cell wall's outer surface (mannoprotein is a highly effective bioemulsifier) and the presence of biopolymer beta-glucan (an emulsifier) in yeast cell walls. Additionally, the microbe-based composition further can comprise biosurfactants in the culture, which are capable of reducing both surface and interfacial tension, and other metabolites (e.g., enzymes, solvents, lactic acid, ethyl acetate, ethanol, etc.) in the culture.

### Growth of Microbes According to the Subject Invention

The subject invention provides methods for cultivation of microorganisms and production of microbial metabolites and/or other by-products of microbial growth. In one embodiment, the subject invention provides materials and methods for the production of biomass (e.g., viable cellular material), extracellular metabolites (e.g. small molecules and excreted proteins), residual nutrients and/or intracellular components (e.g. enzymes and other proteins).

In certain embodiments, a microbe growth facility produces fresh, high-density microorganisms and/or microbial growth by-products of interest on a desired scale. The microbe growth facility may be located at or near the site of application, or at a different location. The facility produces high-density microbe-based compositions in batch, quasi-continuous, or continuous cultivation.

In certain embodiments, the microbe growth facilities of the subject invention can be located at or near the location where the microbe-based product will be used (e.g., at or near an oil well) For example, the microbe growth facility may be less than 300, 250, 200, 150, 100, 75, 50, 25, 15, 10, 5, 3, or 1 mile from the location of use.

The microbe growth facilities can produce fresh, microbe-based compositions, comprising the microbes themselves, microbial metabolites, and/or other components of the broth in which the microbes are grown. If desired, the compositions can have a high density of vegetative cells or a mixture of vegetative cells, spores, conidia, mycelia and/or other microbial propagules. Advantageously, the compositions can be tailored for use at a specified location. In one embodiment, the microbe growth facility is located on, or near, a site where the microbe-based products will be used.

Advantageously, in preferred embodiments, the methods of the subject invention harness the power of naturally-occurring local microorganisms and their metabolic by-products to improve oil production, transmission and/or refining. Local microbes can be identified based on, for example, salt tolerance, ability to grow at high temperatures, and the use of genetic identification of the sequences described herein.

The microbe growth facilities provide manufacturing versatility by their ability to tailor the microbe-based products to improve synergies with destination geographies. The microbe growth facilities may operate off the grid by utilizing, for example, solar, wind and/or hydroelectric power. Thus, the microbe-based compositions can be produced in remote locations.

The growth vessel used for growing microorganisms can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of microbes in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of the liquid, and air spargers for supplying bubbles of gas to the liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, isopropyl, propanol, butanol, pentanol, hexanol, isobutanol, and/or glycerol; fats and oils such as soybean oil, rice bran oil, canola oil, olive oil, corn oil, sesame oil, and/or linseed oil; etc. These carbon sources may be used independently or in a combination of two or more.

In one embodiment, the method comprises use of two carbon sources, one of which is a saturated oil selected from canola, vegetable, corn, coconut, olive, or any other oil suitable for use in, for example, cooking. In a specific embodiment, the saturated oil is 15% canola oil or discarded oil that has been used for cooking.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included, e.g., L-Alanine.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride and/or sodium carbonate. These inorganic salts may be used independently or in a combination of two or more.

In some embodiments, the method for cultivation may further comprise adding additional acids and/or antimicrobials in the liquid medium before and/or during the cultivation process. Antimicrobial agents or antibiotics are used for protecting the culture against contamination.

Additionally, antifoaming agents may also be added to prevent the formation and/or accumulation of foam when gas is produced during cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the liquid medium may be necessary.

The method and equipment for cultivation of microorganisms and production of the microbial by-products can be performed in a batch, quasi-continuous, or continuous processes.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesriable bacterial growth.

In one embodiment, the subject invention provides methods of producing a microbial metabolite by cultivating a microbe strain of the subject invention under conditions appropriate for growth and production of the metabolite; and, optionally, purifying the metabolite. In a specific embodiment, the metabolite is a biosurfactant. The metabolite may also be, for example, ethanol, lactic acid, beta-glucan, proteins, amino acids, peptides, metabolic intermediates, polyunsaturated fatty acids, and lipids. The metabolite content produced by the method can be, for example, at least 20%, 30%, 40%, 50%, 60%, 70%, 80%, or 90%. The biomass content of the fermentation medium may be, for example from 5 g/l to 180 g/l or more. In one embodiment, the solids content of the medium is from 10 g/l to 150 g/l.

The microbial growth by-product produced by microorganisms of interest may be retained in the microorganisms or secreted into the growth medium. In another embodiment, the method for producing microbial growth by-product may further comprise steps of concentrating and purifying the microbial growth by-product of interest. In a further embodiment, the medium may contain compounds that stabilize the activity of microbial growth by-product.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a microbe-free medium or contain cells, spores, mycelia, conidia or other microbial propagules. In this manner, a quasi-continuous system is created.

Advantageously, the methods of cultivation do not require complicated equipment or high energy consumption. The microorganisms of interest can be cultivated at small or large scale on site and utilized, even being still-mixed with their media. Similarly, the microbial metabolites can also be produced at large quantities at the site of need. Because, in certain embodiments, the microbe-based products can be generated locally, without resort to the microorganism stabilization, preservation, storage and transportation processes of conventional microbial production, a much higher density of live microbes, spores, mycelia, conidia or other microbial propagules can be generated, thereby requiring a smaller volume of the microbe-based product for use in the on-site application or which allows much higher density microbial applications where necessary to achieve the desired efficacy. This allows for a scaled-down bioreactor (e.g., smaller fermentation tank, smaller supplies of starter material, nutrients and pH control agents), which makes the system efficient. Local generation of the microbe-based product also facilitates the inclusion of the growth broth in the product. The broth can contain agents produced during the fermentation that are particularly well-suited for local use.

Locally-produced high density, robust cultures of microbes are more effective in the field than those that have undergone vegetative cell stabilization, have been sporulated or have sat in the supply chain for some time. The microbe-based products of the subject invention are particularly advantageous compared to traditional products wherein cells, spores, mycelia, conidia and/or other microbial propagules have been separated from metabolites and nutrients present in the fermentation growth media. Reduced transportation times allow for the production and delivery of fresh batches of microbes and/or their metabolites at the time and volume as required by local demand.

Advantageously, local microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell- and/or propagule-count product and the associated broth and metabolites in which the microbes are originally grown.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### Preparation of Microbe-based Products

One microbe-based product of the subject invention is simply the fermentation medium containing the microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

The microorganisms in the microbe-based product may be in an active or inactive form. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium (e.g., broth or solid substrate) resulting from the microbial growth can be removed from the growth vessel and transferred via, for example, piping for immediate use.

In one embodiment, the microbe-based product is simply the growth by-products of the microorganism. For example, biosurfactants produced by a microorganism can be collected from a submerged fermentation vessel in crude form, comprising, for example about 50% pure metabolite in liquid broth.

In other embodiments, the microbe-based product (microbes, medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting, for example, the yeast fermentation product, from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Examples of such additives include surfactants, emulsifying agents, lubricants, buffering agents, solubility controlling agents, pH adjusting agents, preservatives, stabilizers and ultra-violet light resistant agents.

In one embodiment, the product may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt as polyprotic acid such as sodium bicarbonate or carbonate, sodium sulfate, sodium phosphate, sodium biphosphate, can be included in the formulation.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Microbial Strains

The microorganisms useful according to the subject invention can be, for example, bacteria, yeast and/or fungi. In one embodiment, the composition comprises a yeast, a fungus and a bacterium. The microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In some embodiments, the microbes are “over-producers” of a particular desirable metabolite, such as, for example, an enzyme, solvent or biosurfactant. For example, the microbes can produce at least 10%, 25%, 50%, 100%, 2-fold, 5-fold, 7.5 fold, 10-fold, 12-fold, 15-fold or more compared to other microbial strains.

In some embodiments, the microorganism is a yeast and/or fungus. Examples of yeast and fungus species suitable for use according to the current invention, include, but are not limited to, Acaulospora, Aspergillus, Aureobasidium (e.g., A. pullulans), Blakeslea, Candida (e.g., C. albicans, C. apicola), Debaryomyces (e.g., D. hansenii), Entomophthora, Fusarium, Hanseniaspora (e.g., H. uvarum), Hansenula, Issatchenkia, Kluyveromyces, Mortierella, Mucor (e.g., M. piriformis), Penicillium, Phythium, Phycomyces, Pichia (e.g., P. anomala, P. guilliermondii, P. occidentalis, P. kudriavzevii), Pseudozyma (e.g., P. aphidis), Rhizopus, Saccharomyces (S. cerevisiae, S. boulardii sequela, S. torula), Starmerella (e.g., S. bombicola), Torulopsis, Thraustochytrium, Trichoderma (e.g., T. reesei, T. harzianum, T. virens), Ustilago (e.g., U. maydis), Wickerhamomyces (e.g., W. anomalus), Williopsis, Zygosaccharomyces (e.g., Z. bailii).

In one embodiment, the microorganism is any yeast known as a “killer yeast.” As used herein, “killer yeast” means a strain of yeast characterized by its secretion of toxic proteins or glycoproteins, to which the strain itself is immune. The exotoxins secreted by killer yeasts are capable of killing other strains of yeast, fungi, or bacteria. Killer yeasts can include, but are not limited to, Wickerhamomyces, Pichia, Hansenula, Saccharomyces, Hanseniaspora, Ustilago Debaryomyces, Candida, Cryptococcus, Kluyveromyces, Torulopsis, Williopsis, Zygosaccharomyces and others.

In one embodiment, the composition comprises a Pichia yeast, such as, for example, P. occidentalis or P. kudriavzevii. In a specific embodiment, the yeast is a unique strain of P. occidentalis that was selected for enhanced enzymatic activity (i.e., over-production of enzymes) and viscosity-reducing capabilities.

In one embodiment, the composition comprises a Trichoderma fungus, such as, for example, T. harzianum. Trichoderma can produce useful metabolites, such as, for example, glycolipid biosurfactants, to help with reduction of oil viscosity.

In certain embodiments, the microorganisms are bacteria, including Gram-positive and Gram-negative bacteria. The bacteria may be, for example, Agrobacterium (e.g., A. radiobacter), Arthrobacter (e.g., A. radiobacter), Azomonas spp., Azotobacter (A. vinelandii, A. chroococcum), Azospirillum (e.g., A. brasiliensis), Bacillus (e.g., B. amyloliquifaciens, B. firmus, B. laterosporus, B. licheniformis, B. megaterium, B. mucilaginosus, B. subtilis), Beijerinckia spp., Bradyrhizobium (e.g., B. japanicum, and B. parasponia), Clavibacter (e.g., C. xyli subsp. xyli and C. xyli subsp. cynodontis), Clostridium (C. butyricum C. tyrobutyricum, C. acetobutyricum, Clostridium NIPER 7, and C. beijerinckii), Cronobacter (e.g., C. sakazakii, C. malonaticus, C. turicensis, C. universalis, C. muytjensii, C. dublinensis, C. condimenti), Cyanobacteria spp., Derxia spp., Erwinia (e.g., E. carotovora), Escherichia coli, Frateuria (e.g., F. aurantia), Klebsiella spp., Microbacterium (e.g., M. laevaniformans), Pantoea (e.g., P. agglomerans), Nocardia spp., Pantoea (e.g., P. agglomerans), Pseudomonas (e.g., P. aeruginosa, P. chlororaphis subsp. aureofaciens (Kluyver), P. putida), Ralslonia (e.g., R. eulropha), Rhizobium (e.g., R. japonicum, Sinorhizobium meliloti, Sinorhizobium fredii, R. leguminosarum biovar trifolii, and R. etli), Rhodospirillum (e.g., R. rubrum), Sphingomonas (e.g., S. paucimobilis), Streptomyces (e.g., S. griseochromogenes, S. qriseus, S.cacaoi, S. aureus, and S. kasugaenis), Streptoverticillium (e.g., S. rimofaciens), and/or Xanthomonas (e.g., X. campestris). In one embodiment, the microorganism is a strain of B. subtilis, such as, for example, B. subtilis var. lotuses B1 or B2, which are effective producers of, for example, surfactin and other lipopeptide biosurfactants. This specification incorporates by reference International Publication No. WO 2017/044953 A1 to the extent it is consistent with the teachings disclosed herein.

In one embodiment, the composition comprises a Cronobacter bacterium, such as, for example, C. sakazakii. Cronobacter spp. have been indicated as having capabilities for degradation of certain hydrocarbon molecules.

In certain embodiments, the microorganisms are biosurfactant-producing strains. Microbial biosurfactants are produced by a variety of microorganisms such as bacteria, fungi, and yeasts. Exemplary biosurfactant-producing microorganisms include Starmerella spp. (S. bombicola), Pseudomonas spp. (P. aeruginosa, P. putida, P. florescens, P. fragi, P. syringae); Flavobacterium spp.; Bacillus spp. (B. subtilis, B. pumillus, B. cereus, B. licheniformis, B. amyloliquefaciens, B. megaterium); Wickerhamomyces spp., Candida spp. (C. albicans, C. rugosa, C. tropicalis, C. lipolytica, C. torulopsis); Rhodococcus spp.; Arthrobacter spp.; Campylobacter spp.; Cornybacterium spp.; Pichia spp.; Saccharomyces (S. cerevisiae, S. boulardii sequela, S. torula); Trichoderma (e.g., T. reesei, T. harzianum, T. virens), as well as others.

Safe, effective microbial biosurfactants reduce the surface and interfacial tensions between the molecules of liquids, solids, and gases. As discussed herein, this activity can be highly advantageous in the context of oil recovery.

Biosurfactants are biodegradable and can be efficiently produced using selected organisms on renewable substrates. Most biosurfactant-producing organisms produce biosurfactants in response to the presence of a hydrocarbon source (e.g. oils, sugar, glycerol, etc.) in the growing media. Other media components such as concentration of iron can also affect biosurfactant production significantly.

Biosurfactants according to the subject invention include, for example, low-molecular-weight glycolipids, lipopeptides, flavolipids, phospholipids, and high-molecular-weight polymers such as lipoproteins, lipopolysaccharide-protein complexes, and polysaccharide-protein-fatty acid complexes.

In one embodiment, the microbial biosurfactant is a glycolipid such as a rhamnolipid, sophorolipids (SLP), trehalose lipid or mannosylerythritol lipid (MEL).

In one embodiment, the microbial biosurfactant is a lipopeptides, such as a surfactin, iturin, fengycin, or lichenysin.

In certain embodiments, the microorganisms are enzyme-producing strains. Microbial enzymes are produced by a variety of microorganisms such as bacteria, fungi, and yeasts.

Enzymes are typically divided into six classes: oxidoreductases, transferases, hydrolases, lyases, isomerases and ligases. Each class is further divided into subclasses and by action. Specific subclasses of enzymes according to the subject invention include, but are not limited to, proteases, amylases, glycosidases, cellulases, glucosidases, glucanases, galactosidases, moannosidases, sucrases, dextranases, hydrolases, methyltransferases, phosphorylases, dehydrogenases (e.g., glucose dehydrogenase, alcohol dehydrogenase), oxygenases (e.g., alkane oxygenases, methane monooxygenases, dioxygenases), hydroxylases (e.g., alkane hydroxylase), esterases, lipases, ligninases, mannanases, oxidases, laccases, tyrosinases, cytochrome P450 enzymes, peroxidases (e.g., chloroperoxidase and other haloperoxidasese), lactases, extracellular enzymes from Aspergillus spp. and other microbial species (e.g., lipases from Bacillus subtilis, B. licheniformis, B. amyloliquefaciens, Serratia marcescens, Pseudomonas aeruginosa, and Staphylococcus aureus; amylases, proteases, and/or lipases from Pichia spp.) and other enzyme-based products known in the oil and gas industry.

Other microbial strains including, for example, other strains capable of accumulating significant amounts of, for example, glycolipid-biosurfactants, enzymes, solvents, acids, hydrocarbon-degrading compounds, and/or other metabolites that have bioemulsifying and surface/interfacial tension-reducing properties (e.g., mannoprotein, beta-glucan) can be used in accordance with the subject invention.

### Methods of Enhanced Oil Recovery

In one embodiment the subject invention provides a method for improving oil recovery by applying to heavy oil, or to an oil recovery site containing heavy oil, the microbe-based composition comprising one or more strains of microorganisms and/or microbial growth by-products. In one embodiment, the oil recovery site can comprise oil sands. The method optionally includes adding nutrients and/or other agents to the site.

In one embodiment, the method further comprises applying an organic ester with the microbe-based composition to enhance viscosity reduction. In a specific embodiment, the organic ester is primary amyl acetate.

The method can be performed in situ by injecting the composition and optional nutrients and/or other agents directly to heavy oil (e.g., in a storage tank), or into an oil reservoir (e.g., into the wellbore). Consequently, a high concentration of metabolites and/or the microorganisms that produce them can be achieved easily and continuously therein. Advantageously, the subject compositions and methods can be used to reduce the viscosity, and/or enhance recovery, of heavy crude oil in “mature” or even “dead” oil reservoirs. In certain embodiments, the method can be used to convert heavy oil to light oil.

The subject invention can be applied during all stages of the chain of operations, including by exploration and production (E&P) operators (e.g., while drilling, while tripping-in or tripping-out of the hole, while circulating mud, while casing, while placing a production liner, while cementing, into onshore and offshore wellbores and/or flowlines), midstream (e.g., into pipelines, tankers, transportation, storage tanks), and in refineries (e.g., heat exchangers, furnaces, distillation towers, cokers, hydrocrackers).

In some embodiments, the amount of composition applied is between 1 and 1,000 BBLS or more, depending on, for example, the heaviness of the crude oil, the size of, for example, the storage tank, or the depth of the reservoir where it is applied.

In some embodiments, the methods comprise determining the measure of the viscosity of the heavy crude oil before and/or after applying the composition. The viscosity can be monitored after application, and more of the composition applied if needed to reach a desired viscosity reduction.

Advantageously, the subject invention can increase the API gravity of crudes, heavy crudes, tar sands and petcokes, as well as reduce or eliminate the need for, and costs associated with, steam injection and other thermal, chemical and mechanical methods of heavy oil extraction. Further reduced or eliminated are the need for diluents (e.g., light or refined crude oil) and water jackets to help move heavy crude through pipelines. Even further, with the reduction of heavy oil viscosity, transportation of oil is less complicated or costly, as the need for tanker trucks and storage tanks is reduced and the use of pipeline transport becomes more feasible.

The microbes can be live (or viable), in spore form, or inactive at the time of application. In preferred embodiments, different microbe strains are cultivated separately, then mixed together prior to, or at the time of, application to the heavy crude oil or oil recovery site.

The crude oil can be incubated with the composition for, e.g., 1 day or longer. The viscosity of crude oil can be decreased by, for example, 20 to 60%, in as little as 8 to 12 hours, and remain at a decreased level for extended periods of time, for example, as long as two weeks (14 days) or longer. Compared with other methods, which often result in a return of the crude oil to its heavy, viscous state shortly after treatment, e.g., overnight, the subject invention provides enhanced methods for improving the characteristics of heavy oil, as well as improving its recovery and/or transportation.

In one embodiment, the method further comprises the step of subjecting the heavy oil to cavitation either immediately prior to, simultaneously with, and/or sometime after the microbe-based composition has been applied to the heavy oil or oil recovery site. The cavitation can be carried out using machinery known in the art, and can comprise, for example, hydrodynamic or ultrasonic methods.

As used herein, “cavitation” in the context of treating heavy oil means the formation, growth, and collapse or implosion of gas or vapor filled bubbles in liquids. Cavitation requires the presence of small and transient microcavities or microbubbles of vapor or gas, which grow and then implode or collapse. During cavitation of heavy oil, a portion of the liquid comprising the heavy oil is in the form of a gas, which is dispersed as bubbles in the liquid portion. The process effectively de-structures the molecular arrangement of heavy hydrocarbons in oil (e.g., asphaltenes, which can form highly associative and cohesive aggregates), thereby reducing its viscosity.

In hydrodynamic cavitation, the liquid comprising the heavy oil is passed through a restriction or cavitation zone, such as, for example, a capillary or nozzle, to increase the velocity of the mixture. The gaseous portion may be present prior to passing the liquid comprising the heavy oil through the cavitation zone and/or such gaseous portion may be produced as a result of the pressure drop that results from passing the liquid comprising the heavy oil through the cavitation zone.

In ultrasonic cavitation, sound waves are propagated into the liquid, resulting in alternating high and low pressure cycles. During the low pressure cycle, high intensity ultrasonic waves create small vacuum bubbles or voids in the liquid. When the bubbles attain a volume at which they can no longer absorb energy, they collapse violently during a high pressure cycle.

The cavitation step according to the subject methods can be applied to heavy crude oil at any point during the oil recovery and transport chain of operation in order to prevent or reduce sedimentation of heavy hydrocarbons in the crude fluids, for example, after recovery from a well and before being placed in a collection tank; during storage; after storage in a collection tank and before being transported in a tanker; during transportation; before the refining process, etc. Cavitation machinery can be attached to a storage tank, tanker truck, pump system, piping, tubing, and/or any other equipment used for transport, transmission and/or storage of crude oil.

Advantageously, the methods can increase the amount of upgraded, usable, and valuable oil products that can be produced from heavy oils, for example, by decreasing the Btu of the heavy oil prior to refining. In other words, because the oil has been treated prior to refining, more useful products such as fuel oils, kerosene, and diesel fuel, and less petcoke, for example, can be produced using less complex refining processes than if the oil were left untreated and highly viscous. Furthermore, in preferred embodiments, the subject invention can be used without increasing the TAN of oil.

In one embodiment, methods are provided for recovering oil from oil sands. Oil sands, tar sands, or bituminous sands, are a type of petroleum deposit comprising either loose sands or partially consolidated sandstone. They can contain a mixture of sand, clay and water, and are typically saturated with dense, highly viscous oil known as bitumen (or tar). To recover oil from oil sands, the microbe-based composition can be applied to the oil sands, increasing the wettability of the sands and allowing for detachment of the oil from the sands. Optionally, heat exchangers or another heat source can be used to warm the process.

According to this method, the sands and other solid particles present in the mixture will settle to the bottom of the mixture, and the oil and other composition liquids can be piped to, for example, a storage tank, where they can further be separated from one another. In one embodiment, the oil sands receive cavitation treatment. In a further embodiment, oil that has been separated from the oil sands is subjected to cavitation treatment.

In one embodiment, the viscosity of the oil recovered from the oil sands can be reduced according to the methods of the subject invention, that is, by applying the subject microbe-based compositions to the oil, optionally followed by subjecting the oil to cavitation.

In one embodiment, the present invention provides methods of improving transportation of heavy crude oil, comprising contacting the oil with the microbe-based composition and optional nutrients and/or other agents. Once the heavy oil viscosity is reduced, heavy oils can be easily transported by pipeline rather than requiring transportation in storage tanks by trucks.

### REFERENCES

PetroWiki. Heavy Oil. SPE International; [updated 19 Jan. 2016; accessed 7 Feb. 2017]. http://petrowiki.org/Heavy_oi1#cite_note-r1-1. (“Heavy Oil” 2016).

1. A composition for reducing the viscosity of oil, wherein the composition comprises one or more microorganisms and/or microbial growth by-products produced by the microorganisms, wherein said microorganisms are selected from Pichia occidentalis. Pichia kudriavzevii, Trichoderma spp., and Cronobacter spp.

2. (canceled)

3. The composition of claim 1, comprising a strain of Pichia occidentalis that has enhanced enzymatic activity and viscosity-reducing capabilities, or a growth product thereof.

4. The composition of claim 1, comprising a Trichoderma spp. fungus, or a growth product thereof.

5. The composition of claim 4, wherein the Trichoderma spp. fungus is T. harzianum.

6. The composition of claim 1, comprising a Cronobacter spp. bacterium or a growth product thereof.

7. The composition of claim 6, wherein Cronobacter spp. bacterium is Cronobacter sakazakii.

8. The composition of claim 1, comprising a mixture of Pichia occidentalis, Trichoderma harzianum, and Cronobacter sakazakii, and/or growth products thereof.

9. The composition of claim 1, wherein the microbial growth by-products comprise one or more of: biosurfactants, enzymes and solvents.

10. The composition of claim 1, further comprising an organic solvent.

11. The composition of claim 10, wherein the organic solvent is primary amyl acetate.

12. A method for reducing the viscosity of heavy oil, the method comprising applying a composition oc claim 1 to the oil or to an oil recovery site containing heavy oil.

13. The method of claim 12, wherein applying the composition comprises injecting the composition into a wellbore, flowline, or oil tank.

14. The method of claim 12, further comprising administering nutrients for microbial growth.

15. The method of claim 14, wherein the nutrients comprise sources of nitrogen, nitrate, phosphorus, magnesium and/or carbon.

16. The method of claim 12, used to improve oil transmission through an oil field pipe line, tank, casing, tubing, rod, pump, and/or wellbore.

17. The method of claim 12, wherein the composition is produced on-site at a distance not more than 50 miles from the site at which it is used.

18. The method of claim 12, further comprising the step of subjecting the oil to cavitation.
